(19) United States
(12) Patent Application Publication
(10) Pub. No.: US 2020/0324425 A1
(43) Pub. Date: Oct. 15, 2020

(54) DIGITAL COMMUNICATION ON RAZOR CARTRIDGE, HANDLE AND CHARGING BASE

(71) Applicant: Louis D. Tomassetti, Pompano Beach, FL (US)

(72) Inventor: Louis D. Tomassetti, Pompano Beach, FL (US)

(21) Appl. No.: 16/845,287

(22) Filed: Apr. 10, 2020

Related U.S. Application Data
(60) Provisional application No. 62/832,007, filed on Apr. 10, 2019.

(51) Int. Cl. B26B 21/40; H04W 4/80; H04M 1/21; B26B 21/22; B26B 21/52; B26B 21/48; A61L 2/04

(52) U.S. Cl. CPC B26B 21/405; H04W 4/80; H04M 1/21; B26B 21/225; B26B 21/521; C21D 1/26; B26B 21/48; B26B 21/4087; B26B 21/4037; A61L 2/04; B26B 21/526

(57) ABSTRACT

A razor includes a blade cartridge with multiple blades removably and pivotally attaches to a handle. The blade cartridge and handle include microprocessors for wireless communication between the handle and blade cartridge, via near field communication or RFID. A charging base allows for charging a rechargeable power source in the handle and is further adapted to communicate with a smartphone, computer or other electronic device via wired and/or wireless communication. A mobile application on the smartphone is able to communicate with the charging base, while also providing for wired or wireless communication with remote servers/cloud infrastructure and/or the Internet.

### BACKGROUND OF THE INVENTION

This non-provisional patent application is based on provisional patent application Ser. No. 62/832,007 filed on Apr. 10, 2019.

### FIELD OF THE INVENTION

The present invention relates to razors for shaving and, more particularly, to a wet shave razor having a microprocessor(s) for collecting, storing and wirelessly transferring data.

### DISCUSSION OF THE RELATED ART

Razors for shaving, and particularly wet shave razors, have been known and in use by a large percentage of the world population for many years. And, while there have been continued improvements made to razors over the years, the shaving industry has not seen any significant technological improvements to wet shave razors. Even electric razors have not been improved to any significant degree over the last 30 years.

In todays age of wireless communication, smart technology and artificial intelligence, the current state of the art in shave technology is antiquated to say the least. Simple implements, such as glide strips, multiple blades, pivoting action of the blade cartridge and ergonomically shaped handles are touted by razor manufacturers as the latest advancements, making their razors the best a customer can buy in the marketplace. And yet compared to the technology currently available in other industries, the most expensive wet shave razors and electric shave razors in stores today seem old-fashioned and outdated.

Accordingly, there remains an urgent need in the shaving industry for a technologically advanced razor that provides the most up-to-date electronic and wireless technology to best serve the modern consumer.

### SUMMARY OF THE INVENTION

The present invention is directed to a razor that has microprocessors for collecting, storing and transferring data. The razor includes a blade cartridge with multiple blades that removably and pivotally attaches to a handle. The razor includes a microprocessor embedded within the blade cartridge that allows for unique identification of the cartridge, as well as providing for memory to store data. The handle of the razor includes a microprocessor that allows for bidirectional communication between the handle and the charging base. The handle further contains a rechargeable power source. The razor is placed on a charging base to charge the rechargeable power source in the handle and/or blade cartridge. The charging base is structured to communicate with a smartphone, computer or other electronic device via wireless communication. A microphone in the handle allows for measuring noise of the blade cartridge traveling over the skin surface to calculate wearing of the blades. By sharing this data with a cloud-based service, a notification can be sent to the user indicating that it is time to replace the blade cartridge.

### BRIEF DESCRIPTION OF THE DRAWINGS

For a fuller understanding of the nature of the present invention, reference should be made to the following detailed description taken in conjunction with the accompanying drawings in which:

FIG. 1 is a side elevational view, shown schematically, and illustrating one embodiment of the razor and charging base of the present invention;

FIG. 2 is a side elevational view, shown schematically, and illustrating the razor and charging base of FIG. 1 and further illustrating communication between the charging base and a smartphone and a computer which communicate and transfer data to a remote server, a cloud-based service and/or the Internet;

FIG. 3 is a side elevational view, shown schematically, and illustrating a further embodiment of the razor of the present invention; and

FIG. 4 is a side elevational view, shown schematically, and illustrating a further embodiment of the charging base including a cover and a heater for heating the blades.

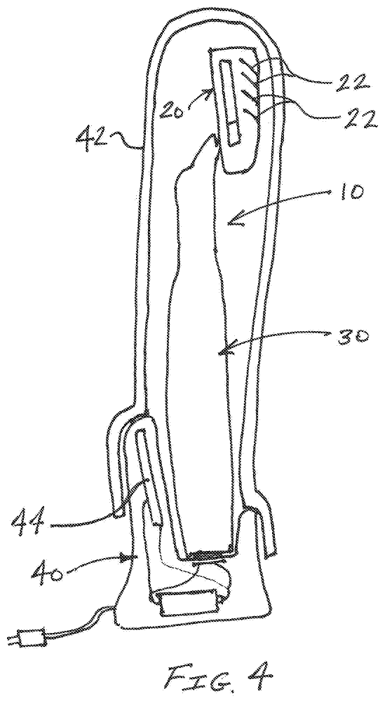

Like reference numerals refer to like parts throughout the several views of the drawings.

### DETAILED DESCRIPTION OF THE PREFERRED EMBODIMENTS

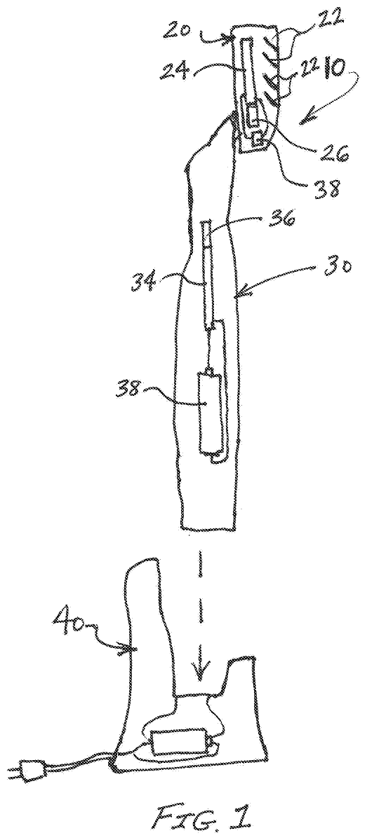

Referring to the several views of the drawings, the razor of the present invention is shown schematically and is generally indicated as 10. The razor 10 has a blade cartridge 20 that includes at least one blade 22 and preferably multiple blades 22. The razor cartridge is pivotally and removably attached to a handle 30. The razor 10 includes microprocessors for collecting, storing and transferring data. In one embodiment, as shown in FIG. 1, the blade cartridge 20 includes a microprocessor 24 that uses near field NFC (near field communication) and/or RFID (radiofrequency identification). The microprocessor 24 is embedded into the cartridge 20 and allows for unique identification of the cartridge 20 as well as memory. The microprocessor 24 has an NFC chip 26. The handle 30 of the razor 10 also includes a microprocessor 34 with an NFC communication chip 36 as well for wireless communication with the microprocessor 24 in the blade cartridge. Additionally, a wireless charger device 40 (i.e., charging base) allows for bidirectional communication between the handle 30 and the charging base 40. This provides for the ability to synchronize data between the blade cartridge 20, the handle 30 and the charging base 40.

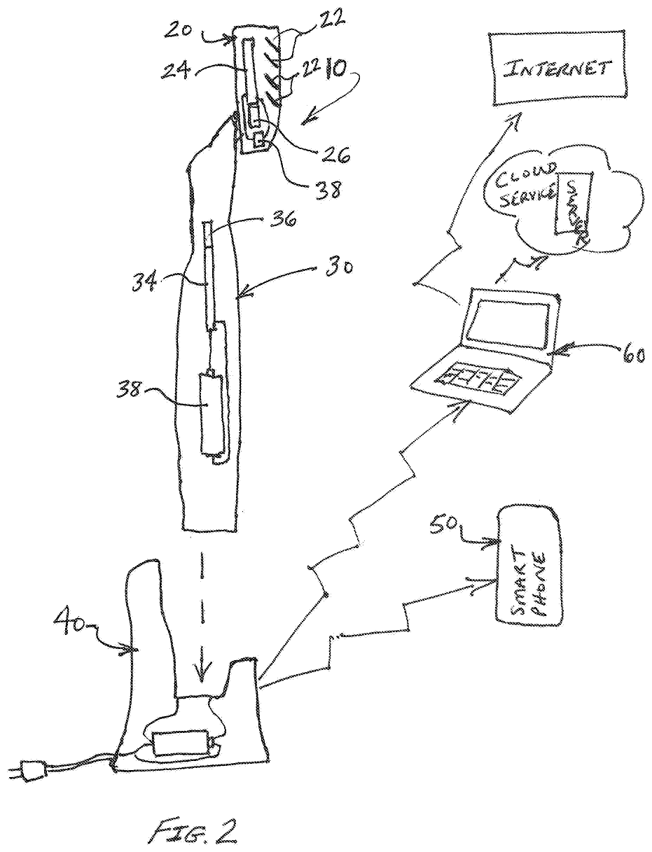

In a further embodiment, as shown in FIG. 2, the charging base 40, in addition to providing the function of a wireless charger for charging a power source 38 in the handle 30 and/or blade cartridge, also includes wired and/or wireless communication components and functionality to allow wireless communication with a smart phone 50, computer 60 or other electronic device. The smart phone 50 will have a computer application (i.e., mobile app) to communicate with the charging base 40 (“smart base”), while also providing a means for wired or wireless communication with remote servers/cloud infrastructure and/or the Internet. In another embodiment, the handle communicates with the smart phone directly via low energy wireless communication.

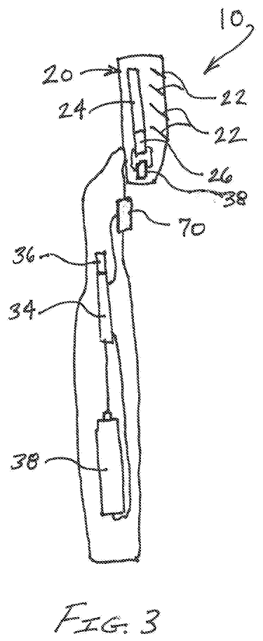

In yet a further embodiment, as seen in FIG. 3, a microphone 70 is provided in the handle 30 of the razor 10. The microphone 70 receives noise as the user shaves. As the blades 22 move along the skin surface, the cutting of the hairs creates a noise. By measuring the envelope of the noise, as well as the frequency of the noise/pitch of the noise, the speed of the shaving strokes can be determined. Knowing the speed of the shaving strokes and the time that each stroke takes, the distance of travel of the blades 22 across the skin surface is easily calculated. The calculated distance is metered (i.e., added to the sum total of previously calculated shaving stroke distances) for each blade cartridge. By sharing this data with a cloud-based service, a notification can be sent to the user of the razor 10 indicating that the cartridge 20 (i.e., useful life of the blades) has expired or is about to expire, along with instructions that the user will need to replace the blade cartridge 20 to ensure a smooth shave.

This allows a razor supply service to trace the supply chain. Because each cartridge has a unique ID, by having the database of those cartridges beforehand, the supply service can always trace them to the end user. This allows a lot of insight to the supply chain and to the shaving habits of the user. For example, a smart phone application can trace how often the user shaves and can remind the user when they need a shave.

Additionally, the user can re-order the blade cartridges either automatically via the smart phone application or manually. Since each blade cartridge has a unique ID, each blade cartridge can be identified to a specific user. The user can then be alerted if they are about to use someone else's blade cartridge, which may be beneficial for sanitary reasons.

In addition to communication, at least one embodiment of the charging base 40 can have HMI (human machine interface). Additionally, the blade cartridges 20 can be heated to sanitize the blades 22 and anneal the blade material because when the blade material is annealed you have better cutting properties. If the charging base 40 is provided with a heater device 44 and is covered, we can controllably heat up/overheat the blades 22 to sanitize the blades 22 by killing the bacteria above 180-200 degrees Fahrenheit (the blades 22 and cartridge 20 could potentially be heated to 300 degrees Fahrenheit because the plastic would not melt up to this temperature). This will prolong the life of the blades 22.

Annealing the metal: aligning all the lattices in the metal. When the metal blades 22 are heated (e.g., above 200 degrees Fahrenheit) and then slowly cooled down, all the lattices in the metal in the structure are aligned, instead of being randomly arranged. The benefit of the lattices aligned is better cutting properties. Instead of being jagged edges on the blade 22, you have smooth edges. Another benefit is it helps to make the blades 22 last longer. Annealing softens it slightly so the lattices will be aligned. Another benefit of heating the blades 22 is killing the bacteria to thereby sanitize the blades and burn away whiskers, skin and oils that remain on the blades surfaces after shaving. This may require heating the blades 22 to 350-400 degrees Fahrenheit. To protect the user, the cover 42 of the charging base 40 can be provided with a safety mechanism which will prevent removal of the cover 42 when the blades 22 are above a certain temperature.

While the present invention has been shown in accordance with several preferred and practical embodiments, it is recognized that departures from the instant disclosure are fully contemplated within the spirit and scope of the present invention which is not to be limited except as defined in the following claims as interpreted under the Doctrine of Equivalents.

What is claimed is:

1. A razor comprising:
a handle;
a blade cartridge pivotally and removably attachable to the handle and including a plurality of blades and a first microprocessor for collecting, storing and transferring data, and the microprocessor uniquely identifying the blade cartridge and the data; and
a rechargeable power source for powering the microprocessor.

2. The razor as recited in claim 1 further comprising:
a second microprocessor in the handle, and the first microprocessor and the second microprocessor both structured for communicating with each other.

3. The razor as recited in claim 2 wherein the first microprocessor and the second microprocessor communicate with each other using near field communication.

4. The razor as recited in claim 3 wherein the first microprocessor includes a near field communication chip and the second microprocessor includes a near field communication chip, and the near field communication chips allowing wireless communication between the first and second microprocessors, including transferring and receiving the data, and storing the data.

5. The razor as recited in claim 4 further comprising:
a charging base for charging the rechargeable power source.

6. The razor as recited in claim 5 wherein the charging base is structured and disposed for wirelessly communicating with the first microprocessor in the blade cartridge and the second microprocessor in the handle.

7. The razor as recited in claim 6 wherein the charging base is further structured and disposed for communicating with an electronic computer device.

8. The razor as recited in claim 7 wherein the electronic computer device communicates with a remote server.

9. The razor as recited in claim 7 wherein the electronic computer device communicates with a cloud-based service.

10. The razor as recited in claim 7 wherein the electronic computer device communicates with the Internet.

11. The razor as recited in claim 7 wherein the electronic computer device is a smartphone.

12. The razor as recited in claim 5 wherein the charging base further includes a cover for covering and containing the razor while the razor is seated on the charging base.

13. The razor as recited in claim 12 wherein the charging base further includes a heater for heating the plurality of blades of the blade cartridge to a temperature of at least 200 degrees Fahrenheit.

14. The razor as recited in claim 13 wherein the heater in the charging base is structured for heating the plurality of blades to a temperature of at least 200 degrees Fahrenheit to anneal the plurality of blades and improve the cutting performance of the plurality of blades, while also extending the useful life of the plurality of blades.

15. The razor as recited in claim 13 wherein the charging base and the heater are structured for heating the temperature of the plurality of blades to at least 180 degrees Fahrenheit to sanitize the plurality of blades.

16. The razor as recited in claim 1 further comprising:
a microphone for receiving noise created by the blade cartridge and the plurality of blades moving along the skin surface of a user of the razor while shaving, and for transferring noise data corresponding to the received noise to the microprocessor to determine speeds of the shaving strokes and distances traveled by the plurality of blades along the skin surface.
